(19) United States
(12) Patent Application Publication
(10) Pub. No.: US 2024/0165185 A1
(43) Pub. Date: May 23, 2024

(54) COMBINATION OF A MYRTLE EXTRACT AND A TRIPTERYGIUM WILFORDII EXTRACT FOR COMBATING C. ACNES-INDUCED INFLAMMATION

(71) Applicant: PIERRE FABRE DERMO-COSMETIQUE, BOULOGNE-BILLANCOURT (FR)

(72) Inventors: Sandrine BESSOU-TOUYA, CASTRES (FR); Lucile GARIDOU, CASTRES (FR); Céline MIAS, AUZEVILLE TOLOSAN (FR)

(73) Assignee: PIERRE FABRE DERMO-COSMETIQUE, BOULOGNE-BILLANCOURT (FR)

(21) Appl. No.: 18/281,088

(22) PCT Filed: Mar. 10, 2022
(86) PCT No.: PCT/FR2022/050431

§ 371 Date: Sep. 8, 2023

(30) Foreign Application Priority Data
Mar. 10, 2021 (FR) 2102343

(51) Int. Cl. A61K 36/37; A61K 8/9789; A61K 31/352; A61K 31/56; A61K 36/61; A61P 17/10; A61P 29/00; A61Q 19/00; C12N 5/04

(52) U.S. Cl. CPC A61K 36/37; A61K 8/9789; A61K 31/352; A61K 31/56; A61K 36/61; A61P 17/10; A61P 29/00; A61Q 19/00; C12N 5/04; A61K 2236/10; A61K 2236/35; A61K 2236/39; A61K 2236/51

(57) ABSTRACT

The present invention relates to a combination comprising a myrtle extract and an extract of Tripterygium wilfordii, in particular for use in the treatment of C. acnes-induced inflammation and in the treatment of acneic skin.

### FIELD OF THE INVENTION

The present invention relates to a novel combination comprising a myrtle extract and an extract of Tripterygium wilfordii, as well as compositions comprising this combination, in particular for their use in the field of acne, and in particular in the treatment of inflammation induced by C. acnes.

### PRIOR ART

Acne is common multi-factor skin condition of the hair follicles and sebaceous glands, causing formation of comedones, affecting the face, scapular region, arms and intertriginous regions. It is the main cause of the most frequent skin conditions. It is important not to trivialise this condition and to treat it correctly, because it can have disabling psychosocial consequences, in particular due to the formation of scars.

There are many forms of acne, the factor common to all of these being the attack on the pilosebaceous follicles. These include, for example, common acne, acne conglobata, keloid acne of the neck, acne medicamentosa, recurrent military acne, necrotic acne, neonatal acne, premenstrual acne, occupational acne, senile acne and solar acne.

Common acne or acne vulgaris, also called juvenile polymorphic acne, is the most common and comprises four stages:
Stage 1 corresponds to comedonal acne and is characterised by a large number of open and/or closed comedones and microcysts;
Stage 2, or papulopustular acne, is of light to moderate severity and is characterised by the presence of open and/or closed comedones, microcysts, but also red papules and pustules. It mainly affects the face and leaves some scars;
Stage 3, or papulocomedonal acne, is more severe and extends to the back, thorax and shoulders. It is accompanied by a large number of scars;
Stage 4, or nodulocystic acne is accompanied by a large number of scars. It presents nodules and also large and painful purple pustules.

In the lightest form, acne affects almost all human beings. Its frequency is highest at the age of puberty, but it can appear for the first time from the age of 7 to 9 years old, and up to ages in excess of 40 years old. Acne is frequently still suffered after the age of 25 years old. Moreover, acne affects both men and women equally. During puberty, under the influence of hormonal secretions and in particular androgens, but also combined with various external factors, an overproduction of sebum is observed, called hyperseborrhea. In subjects predisposed to acne, this environment is conducive to the growth of the key acne bacterium, Cutibacterium acnes (C. acnes) (formerly called Propionibacterium acnes). This bacterium metabolises skin triglycerides into irritating fatty acids via lipases which attack the wall of the follicle and the surrounding dermis, also produces various enzymes and chemo-attractants of immune phagocytic cells, and stimulates the production of proinflammatory cytokines by various types of cells (in particular sebocytes, keratinocytes and monocytes) which aggravate the inflammation. It has long been thought that combating this bacterial species was the priority in the treatment of acne. Various topical antimicrobials are still widely used today (benzoyl peroxide, erythromycin, triclosan). Recently, however, researchers have understood that the goal is not to eradicate C. acnes which is a commensal bacterium, necessary for tissue homeostasis, but to re-establish an equilibrium because acne is associated with a loss of diversity of phylotypes of C. acnes, with a predominance of the pathogenic phylotype IA 1 . Contrary to what has long been thought, acne is not associated with multiplication of C. acnes but with a change in the ratio of phylotypes and a loss of the richness of these phylotypes.

Similarly, a more or less prolonged systemic antibiotic therapy was sometimes combined with the treatment, depending on the severity of the condition (tetracyclines, doxycycline). Today, dermatologists will turn more towards local anti-inflammatories, seboregulators.

Frequent failures of all these treatments have been observed, often due to a large proportion of resistant strains of C. acnes. This resistance can be the consequence of an organisation of bacterial populations into a biofilm. Biofilms are bacterial cell communities incorporated in an extracellular matrix excreted by microorganisms, composed of sugar polymers and called glycocalyx. Sessile bacteria (associated with the biofilm) are phenotypically and physiologically different from planktonic (free) bacteria. Studies have confirmed the ability of C. acnes to form biofilms equally well in vitro (Holmberg et al., Clin. Microbiol. Infect. 2009, 15, 787-795) as in vivo, on medical devices (Craig et al., J. Am. Acad. Dermatol. 2007, 722-30 724).

This bacterium plays a pivotal role in acne, in particular by stimulating the local inflammatory response (Dagnelie et al., Journal of the European Academy of Dermatology 2019, 33(12), 2340-2348). Contrary to that which was long been believed, C. acnes does not proliferate in the pilosebaceous follicle of the acne-prone skin, but a loss is observed of the richness/diversity of the various phylotypes of this bacterium with a strong predominance of the phylotype IA1. This phylotype is pro-pathogen and has a strong capacity to organise into a biofilm giving this bacterium a stronger virulence. This virulence can be measured by the quantification of the virulence factors produced by C. acnes.

C. acnes IA1 stimulates the local inflammatory response by acting directly on the pilosebaceous follicle cells and in particular the keratinocytes, sebocytes but also the immune cells: monocytes. It acts by stimulating the production of pro-inflammatory cytokines by these cells and in particular interleukins 6 and 8. This is followed by an immuno-inflammatory cascade and more particularly the recruitment and differentiation of naive CD4+ T lymphocytes into LTH17. These LTH17, recently demonstrated in the pathology of acne, specifically produce interleukin 17, from the sub-clinical stages of acne to the most inflammatory lesions.

Thus, there is still a need to provide more effective treatments of acne, which do not have undesirable side effects for the patient. In particular, there are currently no agents which act both directly on the biofilm of C. acnes and on the inflammatory reactions resulting from it.

### SUMMARY OF THE INVENTION

An object of the invention is to respond to these needs. Indeed, the inventors have unexpectedly shown that the combination of a myrtle extract and an extract of Tripterygium wilfordii has the capacity to reduce the production of inflammatory cytokines, the inflammation being stimulated in the presence of C. acnes. More specifically, the combination according to the invention has the advantage of acting synergistically on the immuno-inflammatory cascade mediated by C. acnes and of thus being useful in the treatment of acne.

An object of the present invention is therefore a combination comprising a myrtle extract and an extract of Tripterygium wilfordii.

Another object of the present invention is a cosmetic or dermatological composition comprising a combination comprising a myrtle extract and an extract of Tripterygium wilfordii, with at least one cosmetically or dermatologically acceptable excipient.

Another object of the present invention is a combination according to the invention, in other words comprising a myrtle extract and an extract of Tripterygium wilfordii, for use thereof in the treatment of inflammation induced by C. acnes.

The invention also relates to the use of a combination according to the invention for preparing a cosmetic or dermatological composition for treating inflammation induced by C. acnes.

The invention also relates to the use of a combination according to the invention in the treatment of inflammation induced by C. acnes.

The invention also relates to a method for treating inflammation induced by C. acnes comprising the administration, to a person in need thereof, of an effective amount of a combination according to the invention.

Another object of the present invention is a combination according to the invention for use thereof in the treatment of acne or acne-prone skin.

The invention also relates to the use of a combination according to the invention for preparing a cosmetic or dermatological composition for treating acne or acne-prone skin.

The invention also relates to the use of a combination according to the invention in the treatment of acne or acne-prone skin.

The invention also relates to a method for treating acne or acne-prone skin, comprising the administration, to a person in need thereof, of an effective amount of a combination according to the invention.

Another object of the present invention is a cosmetic or dermatological composition according to the invention, in other words comprising a combination comprising a myrtle extract and an extract of Tripterygium wilfordii, with at least one cosmetically or dermatologically acceptable excipient, for use thereof in the treatment of inflammation induced by C. acnes.

The invention also relates to the use of a cosmetic or dermatological composition according to the invention for preparing a drug intended for treating inflammation induced by C. acnes.

The invention also relates to the use of a cosmetic or dermatological composition according to the invention in the treatment of inflammation induced by C. acnes.

The invention also relates to a method for treating inflammation induced by C. acnes comprising the administration, to a person in need thereof, of an effective amount of a cosmetic or dermatological composition according to the invention.

Another object of the present invention is a cosmetic or dermatological composition according to the invention for use thereof in the treatment of acne or acne-prone skin.

The invention also relates to the use of a cosmetic or dermatological composition according to the invention for preparing a drug for treating acne or acne-prone skin.

The invention also relates to the use of a cosmetic or dermatological composition according to the invention in the treatment of acne or acne-prone skin.

The invention also relates to a method for treating acne or acne-prone skin, comprising the administration, to a person in need thereof, of an effective amount of a cosmetic or dermatological composition according to the invention.

### DETAILED DESCRIPTION OF THE INVENTION

According to a first aspect, the invention relates to a combination comprising a myrtle extract and an extract of Tripterygium wilfordii.

### Myrtle Extract

Common myrtle, Myrtus communis L., is a shrub of the Myrtaceae family. The myrtle extract according to the present invention is produced, more particularly, from the leaves of Myrtus communis. It preferably involves a non-polar fraction of aerial parts, and more particularly the leaves, of myrtle.

The term “non-polar fraction” shall mean a fraction of a non-polar extract, typically a non-polar extract treated by activated carbon so as to remove chlorophylls.

The term “non-polar extract” shall mean an extract that can be obtained using a non-polar extraction solvent, such as ethyl acetate, isopropyl acetate or a mixture thereof.

According to the invention, the term “aerial parts” shall mean the parts of the plant located above the ground, for example the leaves, stems, petioles and/or inflorescences, in particular the leaves.

The extract according to the present invention can be obtained by extraction using a solvent or mixture of solvents (called extraction solvent) chosen from:
alcohols such as ethanol, methanol, isopropanol,
ketones including acetone and methyl ethyl ketone,
hexane,
methylene chloride
isopropyl ether,
ethyl acetate or isopropyl acetate,
and the mixtures thereof;
   or by extraction using supercritical CO₂.

According to an embodiment, the myrtle extract is obtainable by extraction using isopropyl acetate, preferably from the aerial parts, and in particular the leaves, of Myrtus communis.

Hence, the plant (or any part thereof) is placed in contact with the extraction solvent. Once the extraction has been performed, the plant/extraction solvent mixture is filtered so as to separate the solvent phase from the plant residues (called marc). The marc thus obtained is rinsed, typically with the same solvent as the extraction solvent, and the rinsing solvent thus obtained is mixed with the solvent phase so as to obtain an extraction liquor.

Advantageously, the extraction liquors, obtained after filtering and rinsing of the marc, are bleached by addition of activated carbon, which enables the removal of chlorophylls.

The extract can also be stabilised by addition of an antioxidant, such as butylated hydroxytoluene or alpha tocopherol, in particular in quantities between 0.05 and 1% by weight of dry extract.

The extract according to the present invention has, in particular, the feature of comprising, and in particular being rich in, myrtucommulones and ursolic acid. Myrtucommulones A, B′, D, B, isos (isosemimyrtucommulmone) and S (semimyrtucommulone) are advantageously present in the extract and are, in particular, the main myrtucommulones present.

Advantageously, the total content of myrtucommulones in the extract is between 3 and 10% by weight of dry extract.

The content of ursolic acid is between 10 and 30%, preferably >15% by weight of dry extract.

These molecules carry, at least in part, the activity claimed in the context of the present invention.

In a particular embodiment of the invention, the myrtle extract will preferably be as described in patent application EP 1 112 079, the teaching of which is incorporated in the present application or in accordance with example 1 of the present application. This document, EP 1 112 079, describes the antimicrobial properties of the extract of Myrtus communis and its applications in cosmetic and dermatological compositions.

The myrtle extract as used in the present invention is also the subject matter of patent FR 2 992 862, its anti-biofilm activity with respect to C. acnes being described therein.

### Extract of Tripterygium wilfordii

Tripterygium wilfordii is a medicinal plant belonging to the Celastraceae family. Terpenes are among the most active components of the plant and are mostly located in the roots of the plant.

These include, in particular, pentacyclic triterpenes such as tingenin A (also called tingenone or maytenine), tingenin B (also called 22-beta-hydroxy-tingenone), celastrol, pristimerin and tripterygone. These molecules are described in patent application EP 3 454 875, the teaching of which is incorporated in the present application.

In an embodiment, the extract of Tripterygium wilfordii can be an extract comprising, in particular enriched in, pentacyclic triterpene(s), such as tingenin A, tingenin B and/or celastrol.

The extract can optionally comprise components (including active components) originating from plants other than pentacyclic triterpenes.

The term “pentacyclic triterpene” according to the invention, shall mean a pentacyclic triterpene produced naturally by the cells of the plant Tripterygium wilfordii and in particular celastrol of formula Chem. 1, tingenin A (also called tingenone or maytenine) of formula Chem. II, tingenin B (also called 22-beta-hydroxy-tingenone) of formula Chem. III, pristimerin of formula Chem. IV, and/or tripterygone of formula Chem. V, preferably celastrol, tingenin A and/or tingenin B, in particular celastrol.

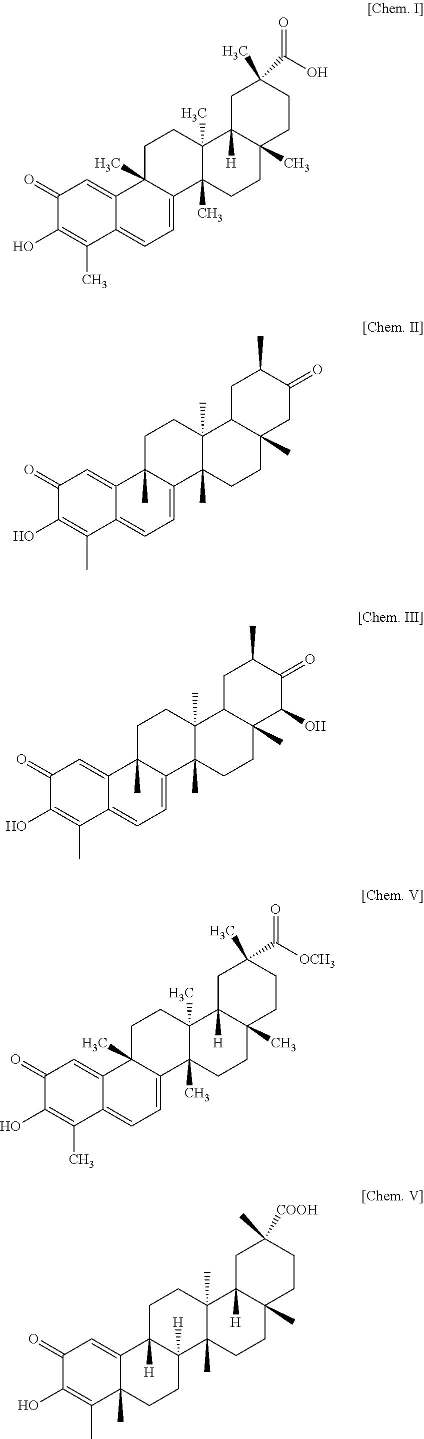

The term “crude extract” shall mean an extract obtained directly from a plant, in this case Tripterygium wilfordii.

The term “enriched extract” of Tripterygium wilfordii, shall mean an extract of Tripterygium wilfordii in which the quantity of pentacyclic triterpene(s), in particular tingenin A, tingenin B and/or celastrol, is greater than 30% by weight, in particular greater than 50% by weight relative to the quantity of pentacyclic triterpenes in a dry crude extract.

In an embodiment, the extract, in particular the enriched extract, of Tripterygium wilfordii for use according to the invention, comprises between 90% and 100% pentacyclic triterpene(s) by weight relative to the total weight of the dry extract, in particular of the enriched dry extract.

The extracts, in particular the crude or enriched extracts, can be obtained from any part of the Tripterygium wilfordii plant, in particular the roots, the seeds or the aerial parts.

Alternatively, the extract of Tripterygium wilfordii can be obtained by plant cell cultures of these plants. In such a case, it will be possible to obtain the extract, in particular, from the supernatant, the suspension or the biomass of said cell cultures, as described, in particular, by Coppede et al., Plant Cell Tiss Organ Cult, 2017, 118, 33-43.

In a preferred embodiment, the extract of Tripterygium wilfordii is an extract comprising, in particular enriched in, pentacyclic triterpene(s) that is obtainable according to the following method:
(i) a proliferation phase of Tripterygium wilfordii cells in a proliferation medium,
(ii) an elicitation phase by addition of an elicitation cocktail to the cell culture obtained in step (i), said elicitation cocktail comprising at least one monocarboxylic compound elicitor and at least one biotic elicitor, and
(iii) the preparation of an extract comprising, in particular enriched in, pentacyclic triterpene(s) from the cell culture obtained in step (ii).

The term “cells of Tripterygium wilfordii” according to the invention, shall mean the cells of any part of the plant: seeds, roots, aerial parts, and in particular aerial parts, and more particularly leaves.

According to the invention, the term “ proliferation phase of cells of Tripterygium wilfordii” shall mean a phase in which the Tripterygium wilfordii cells are in suspension in a proliferation medium and under conditions suitable for their proliferation. These cells will be obtained, in particular, from calluses before their suspension. If necessary, the cell suspensions can be regularly reseeded in order to maintain proliferation conditions.

According to the invention, the term “callus” shall mean a cluster of undifferentiated cells, also called stem cells or meristematic cells.

In the present description, the term “approximately” shall mean that the value concerned can be less than or greater than the indicated value by 10%, notably by 5%, in particular by 1%.

The calluses can be induced by any method known to a person skilled in the art. The calluses according to the invention can, in particular, be obtained in the manner described below. The inducing of calluses by an explant of tissue from a plant part, in particular aerial part, such as a leaf of Tripterygium wilfordii, is well known to a person skilled in the art. The inducing of calluses can be carried out, in particular, by:
obtaining an explant of plant tissue, for example a piece of leaf of approximately 1 cm² in size,
culturing the explant on an agar proliferation medium (for example by adding 4 to 12 g/L of agar, for example approximately 8 g/L of agar, to the proliferation medium according to the invention)
incubating, in particular in the dark, at a temperature of approximately 25-30° C., for example at approximately 27 to 28° C.

### Step (i): Cell Proliferation Phase

A person skilled in the art, familiar with cell cultures of a plant like Tripterygium wilfordii, will easily determine the composition of the proliferation medium necessary for their proliferation.

Preferably, this proliferation medium will enable the proliferation of cells in undifferentiated form, in other words in totipotent forms. Maintaining the undifferentiated form can be achieved, in particular, by the use of particular cytokinins/auxins ratios in the proliferation medium. The proliferation medium can comprise, in particular:
at least one macroelement chosen in particular from NH₄NO₃, KNO₃, CaCl₂·2H₂O, MgSO₄·7H₂O, KH₂PO₄ and a mixture thereof, for example at a total macroelement concentration between 1000 and 9000 mg/L, for example between 3000 and 8000 mg/L of proliferation medium: during the proliferation phase, the total macroelement concentration of the proliferation medium will be, in particular, between 3000 and 5500 mg/L of proliferation medium;
At least one microelement, chosen in particular from KI, H₃BO₃, MnSO₄·4H₂O, ZnSO₄·H₂O, Na₂MoO₄·2H₂O, CuSO₄·5H₂O, CoCl₂·6H₂O, FeSO₄·7H₂O, Na₂EDTA·2H₂O and a mixture thereof, for example at a total microelement concentration between 10 and 200 mg/L, in particular between 50 and 150 mg/L of proliferation medium;
At least one vitamin, in particular chosen from the myo-inositol, nicotinic acid, pyridoxine hydrochloride, thiamine hydrochloride and a mixture thereof, for example at a total vitamin concentration that can range from 0.01 to 3g/L, in particular from 0.05 to 1 g/L of proliferation medium;
At least one amino acid, in particular glycine, for example at a total amino acid 20 concentration that can range from 0.15 to 5 mg/L, in particular between 1 and 4mg/L of proliferation medium: during the proliferation phase, the amino acid concentration of the proliferation medium will be, in particular, between 1 and 2.5 mg/L of proliferation medium;
least one carbon source, in particular sucrose, for example at a total carbon source concentration of 10 to 70 g/L of proliferation medium, for example approximately 30 g/L;
At least one plant hormone (also called plant growth hormone or plant growth factor or plant growth regulator) chosen, in particular, from one or more cytokinins, in particular kinetin and/or 6-furfurylaminopurine, one or more auxins, in particular 2,4-dichlorophenoxyacetic acid (2,4 D) and/or naphthalene acetic acid (NAA), and a mixture thereof. During the proliferation phase, the proliferation medium comprises, in particular, at least one cytokinin and at least one auxin. The plant growth hormones will be added, in particular, to the proliferation medium at a concentration and in a ratio enabling proliferation of the cells in undifferentiated form. They will be chosen in particular from kinetin, 6-furfurylaminopurine, 2,4 D acid, NAA acid and a mixture thereof; in particular chosen from kinetin, 2,4 D acid, NAA acid and a mixture thereof. It may involve, in particular, a mixture of kinetin, 2,4 D acid and NAA acid.
The concentration of auxins will be, in particular, between 0.001 and 10 mg/L of proliferation medium, for example between 0.1 and 3 mg/L of proliferation medium. The concentration of cytokinins will be, in particular, between 0.01 and 0.5 mg/L of proliferation medium, for example between 0.05 and 0.15 mg/L of proliferation medium.
In an embodiment, the hormonal ratio auxins/cytokinins will be between 0.2 to 2.5/0.01 to 0.5, in particular between 1 to 2/0.05 to 0.2, in particular will be approximately 1.5/0.1.
The proliferation medium according to the invention will comprise, in particular, 1.5 mg/L of auxins, in particular 2,4 D acid and NAA acid, and 0.1 mg/L of cytokinins, in particular kinetins.

The proliferation medium will advantageously be sterile and preferably at a pH close to neutrality.

An example of a proliferation medium suitable for the proliferation of cells from a Tripterygium wilfordii plant according to the invention is described, in particular, by Murashige & Skoog (Physiologia Plantarum, 1962, 15: 473-497) or according to example 2 of the present application.

This proliferation medium can, for example, have the following composition (the concentrations are expressed relative to the volume of proliferation medium without cells): Macroelements: NH₄NO₃ at 1650mg/L, KNO₃ at 1900 mg/L, CaCl₂·2H₂O at 440 mg/L, MgSO₄·7H₂O at 370 mg/L, KH₂PO₄ at 170 mg/L; Microelements: KI at 0.83 mg/L, H₃BO₃ at 6.2 mg/L, MnSO₄·4H₂O at 22.3 mg/L, ZnSO₄·H₂O at 6.6 mg/L, Na₂MoO₄·2H₂O at 0.25 mg/L, CuSO₄·5H₂O at 0.025 mg/L, CoCl_{2·6}H₂O at 0.025 mg/L, FeSO₄·7H₂O at 27.8 mg/L, Na₂EDTA·2H₂O at 37.3 mg/L; Vitamins: myo-inositol at 100 mg/L, nicotinic acid at 0.5 mg/L, pyridoxine hydrochloride at 0.5 mg/L, thiamine hydrochloride at 0.5 mg/L; Amino acids: glycine at 2 mg/L; Carbon source: sucrose at 30 g/L; and Plant hormones: NAA acid at 1 mg/L, 2,4 D acid at 0.5 mg/L, kinetin at 0.1 mg/L, the total adjusted to pH 6 before sterilisation, for example by autoclaving for 20 minutes at 121° C. or by filtering on a 0.2 μm filter.

Alternatively, the proliferation medium can have the following composition (the concentrations are expressed relative to the volume of proliferation medium without cells): Macroelements: NH₄NO₃ at 1650 mg/L, KNO₃ at 2500 mg/L, CaCl₂·2H₂O at 440 mg/L, MgSO₄·7H₂O at 370 mg/L, KH₂PO₄ at 130 mg/L; Microelements: KI at 0.41 mg/L, H₃BO₃ at 6.2 mg/L, MnSO₄·4H₂O at 22.3 mg/L, ZnSO₄·H₂O at 7.5 mg/L, Na₂MoO_{4·2}H₂O at 0.25 mg/L, CuSO₄·5H₂O at 0.025 mg/L, CoCl_{2·6}H₂O at 0.025 mg/L, FeSO₄·7H₂O at 19.85 mg/L, Na₂EDTA·2H₂O at 26.64 mg/L; Vitamins: myo-inositol at 50 mg/L, nicotinic acid at 0.25 mg/L, pyridoxine hydrochloride at 0.25 mg/L, thiamine hydrochloride at 0.25 mg/L; Carbon source: sucrose at 30 g/L; and Plant hormones: NAA acid at 0.35 mg/L, 2,4 D acid at 0.575 mg/L, kinetin at 0.083 mg/L.

Alternatively, the proliferation medium can have the following composition (the concentrations are expressed relative to the volume of proliferation medium without cells): Macroelements: NH₄NO₃ at 20 mM, KNO₃ at 19 mM, CaCl₂·2H₂O at 3 mM, MgSO₄·7H₂O at 1.5 mM, KH₂PO₄ at 1.2 mM, KI at 0.005 mM, H₃BO₃ at 0.1 mM, MnSO₄·4H₂O at 0.1 mM, ZnSO₄·H₂O at 0.04 mM, Na₂MoO_{4·2}H₂O at 0.001 mM, CuSO₄·5H₂O at 0.0001 mM, CoCl_{2·6}H₂O at 0.0001 mM, FeSO₄·7H₂O at 0.1 mM, Na₂EDTA·2H₂O at 0.1 mM, myo-inositol at 0.5 mM, nicotinic acid at 0.004 mM, pyridoxine hydrochloride at 0.002 mM, thiamine hydrochloride at 0.0015 mM, glycine at 0.03 mM, sucrose at 87.6 mM, NAA acid at 0.005 mM, 2,4 D acid at 0.002 mM, kinetin at 0.0005 mM.

The seeding of the proliferation medium will be carried out from the suspension of callus cells at a concentration between 20 and 300 g in 1 L of proliferation medium and preferably between 100 and 200 g in 1 L of proliferation medium, for example approximately 150 g in 1 L of proliferation medium.

The proliferation phase will take place under biomass multiplication conditions.

According to the invention, the term “biomass multiplication conditions” shall mean, in particular, the conditions of temperature, duration, stirring and luminosity necessary for the proliferation of cells in suspension. A person skilled in the art being familiar with Tripterygium wilfordii cell cultures will easily determine the biomass multiplication conditions. In an embodiment according to the invention, the step of proliferation of the biomass will be made in the dark, at a temperature between 20 and 35° C., in particular between 27 and 28° C., in particular at approximately 27 or 28° C., in particular while stirring at between 100 and 200 rpm, in particular at approximately 125 rpm (orbital of 22.5 mm) and for a duration between 10 and 30 days, in particular 15 days of culture.

During this step, the cells can be “subcultured” or propagated, for example every 7 to 15 days.

The subculturing of cells is well known to a person skilled in the art, it may consist, in particular, of diluting a part of the cell culture in a new concentrated medium. For example, ⅕^{th} of the culture is suspended in a volume of new medium corresponding to the volume of the initial culture. This allows the cell line to be maintained in liquid medium in a proliferation state.

### Step (ii): Elicitation Phase

After the proliferation phase, the cells contained in phase (i) are elicited by addition of an elicitation cocktail. The proliferation medium to which the elicitation cocktail is added will be called the “elicitation medium”. The elicitation phase is the phase in which the cells are held in a physiological state favouring the biosynthesis of secondary metabolites such as pentacyclic triterpenes.

The production of pentacyclic triterpenes takes place, during the elicitation phase, in the cytosol of the cell and can partially diffuse into the elicitation medium. Within the meaning of the present invention, the elicitation phase corresponds therefore to the production phase (biosynthesis) of pentacyclic triterpenes and more particularly of celastrol, tingenin A and tingenin B.

The elicitation will preferably be made after a proliferation phase of 7 to 21 days, in particular 12 to 20 days, in particular 15, 16 or 17 days (in particular without subculturing). Alternatively, the addition of the elicitation cocktail can be made when the concentration of cells obtained during the proliferation phase is double, in particular greater than double, relative to the initial concentration of cells in the proliferation medium. The addition of the elicitation cocktail can be made, in particular, when the concentration of cells is greater than 200 g/L, for example between 200 and 400 g/L, in particular approximately 300 g/L (in quantity of cells per litre of proliferation medium). In an embodiment, the addition of the elicitation cocktail will be made in a culture for which the concentration of cells changes from 150 to 200 g/L at the start of the proliferation phase, to a concentration between 300 and 400 g/L at the end of the proliferation phase.

Following the proliferation phase, the plant cells have consumed the majority, or even all, of the elements contained in the proliferation medium, and in particular the carbon sources such as sucrose. It may therefore be necessary to re-establish the composition of the medium before or at the same time as adding the elicitation cocktail. In order to re-establish the composition of the medium, it is possible, in particular, to concentrate the cell culture obtained after the proliferation step, for example by decanting or filtering, then adding new proliferation medium to the cells thus obtained. In this case, the cells will be re-suspended in the new proliferation medium so as to obtain a concentration between 200 and 400 g/L, for example between 250 and 350 g/L, in particular approximately 300 g/L (in quantity of cells per litre of proliferation medium).

Alternatively, it is possible to add, to the cell culture, a concentrated mixture enabling the concentrations of the elements of the proliferation medium to be re-established. The concentrated mixture will be added, in particular, just before, just after or at the same time as the elicitation cocktail. For example, it is possible to replace ⅕^{th} of the cell culture by an equivalent volume of the proliferation medium concentrated by a factor of 5.

It is considered that the concentration of elements of the proliferation medium is close or equivalent to zero after 14, 15 or 16 days of proliferation phase. In particular, it is considered that the concentration of mineral elements (more particularly the macroelements and 10 microelements) and carbonaceous elements (more particularly the sources of carbon) is close to or equivalent to zero after 14, 15 or 16 days of proliferation phase.

In an embodiment, the proliferation medium according to the invention at the start of the elicitation phase will comprise, inter alia:
at least one macroelement, in particular chosen from NH₄NO₃, KNO₃, CaCl₂·2H₂O, MgSO₄·7H₂O, KH₂PO₄ and a mixture thereof, for example at a total macroelement concentration between 5000 and 8000 mg/L, preferably greater than 6000 mg/L of proliferation medium, advantageously between 6000 and 8000 mg/L;
At least one microelement, chosen in particular from KI, H₃BO₃, MnSO₄·4H₂O, ZnSO₄·H₂O, Na₂MoO_{4·2}H₂O, CuSO₄·5H₂O, CoCl_{2·6}H₂O, FeSO₄·7H₂O, Na₂EDTA·2H₂O and a mixture thereof, for example at a total microelement concentration between 10 and 200 mg/L, in particular between 50 and 150 mg/L of proliferation medium;
At least one vitamin, in particular chosen from the myo-inositol, nicotinic acid, pyridoxine hydrochloride, thiamine hydrochloride and a mixture thereof, for example at a total vitamin concentration that can range from 0.01 to 3g/L, in particular from 0.05 to 1 g/L of proliferation medium;
At least one amino acid, in particular glycine, for example at a concentration in the proliferation medium between 3 and 4 mg/L of proliferation medium;
At least one carbon source, in particular sucrose, for example at a total carbon source concentration of 10 to 70 g/L of proliferation medium, for example approximately 30 g/L.

In an embodiment, the proliferation medium at the start of the elicitation phase does not comprise, or will comprise a negligible quantity, in particular less than 0.001 g/mL, of cytokinin and auxin.

The proliferation medium during the elicitation phase will advantageously be sterile and preferably at a pH close to neutrality.

During the elicitation phase, the proliferation medium could have, in particular, the following composition: Macroelements: NH₄NO₃ at 2.8 g/L, KNO₃ at 3 g/L, CaCl₂·2H₂O at 0.45 g/L, MgSO₄·7H₂O at 74 mg/L, KH₂PO₄ at 34 mg/L; Microelements: KI at 0.16 mg/L, H₃BO₃ at 6.2 mg/L, MnSO₄·4H₂O at 18.5 mg/L, ZnSO₄·H₂O at 6.6 mg/L, Na₂MoO_{4·2}H₂O at 0.25 mg/L, CuSO₄·5H₂O at 0.025 mg/L, CoCl_{2·6}H₂O at 0.025 mg/L, FeSO₄·7H₂O at 28 mg/L, Na₂EDTA·2H₂O at 37 mg/L; Vitamins: myo-inositol at 250 mg/L, nicotinic acid at 1.7 mg/L, pyridoxine hydrochloride at 1 mg/L, thiamine hydrochloride at 1 mg/L; Amino acids: glycine at 4 mg/L; Carbon source: sucrose at 30 g/L.

According to the invention, the term “elicitation cocktail” shall mean a cocktail enabling the stopping of cell division. This elicitation cocktail comprises at least one monocarboxylic compound elicitor and at least one biotic elicitor. The elicitation cocktail is introduced into the culture medium using, for example, concentrated parent solutions.

According to the invention, the term “monocarboxylic compound elicitor” shall mean, more particularly, an elicitor chosen from the group comprising, preferably consisting of, 5-chloro salicylic acid, salicylic acid, acetylsalicylic acid, a methyl ester, in particular methyl jasmonate, and a mixture thereof. In an embodiment according to the invention, the monocarboxylic compound elicitor is methyl jasmonate, salicylic acid and/or 5-chloro salicylic acid, in particular methyl jasmonate. The monocarboxylic compound elicitor will be added, in particular, in such a way as to obtain a final concentration between 0.005 and 0.1 g/L, in particular between 0.01 and 0.05 g/L of elicitation medium, in particular between 0.002 and 0.004 g/L of elicitation medium.

According to the invention, the term “biotic elicitor” shall mean more particularly a biotic elicitor chosen from the group comprising, preferably consisting of, N-acetylaminoglucosamine, in particular chitin, chitosan, extracts of microorganisms or fungi, oligosaccharides (polysaccharides, pectins, cellulose). In an embodiment, the biotic elicitor is chitin. Chitin is a linear polymer with repeating unit: beta-1,4 N-acetyl D-glucosamine. The biotic elicitor will be he added, in particular, in such a way as to obtain a final concentration between 0.05 and 50 g/L of elicitation medium, for example from 0.1 to 10 g/L, for example 0.5 to 7 g/L, in particular from 1 to 5 g/L of elicitation medium.

In an embodiment, the elicitation cocktail comprises methyl jasmonate, in particular at a final concentration in the elicitation medium between 0.002 and 0.005 g/L, and chitin, in particular at a final concentration between 1 and 4 g/L of elicitation medium.

In an embodiment, the elicitation cocktail according to the invention will further comprise at least one cell differentiation factor for plant cells and/or at least one precursor of the terpene synthesis path.

According to the invention, the “cell differentiation factor for plant cells ” can be chosen, in particular, from the group comprising, preferably consisting of, a cytokinin, in particular benzylaminopurine, abscisic acid, kinetin, thidiazuron, 6-γ,γ-dimethylallylaminopurine (or isopentenyladenine) or zeatin, a gibberellin and a mixture thereof, in particular benzylaminopurine and/or 6-γ,γ-dimethylallylaminopurine, in particular 6-γ,γ-dimethylallylaminopurine.

According to the invention, the “terpene synthesis precursor” will be chosen, in particular, from the group comprising, preferably consisting of: sodium pyruvate, potassium pyrophosphate, mevalonic acid, geraniol, farnesol, isopentenyl, dimethylallyl, including their pyrophosphated forms, sodium acetate, pyruvic acid and the mixtures thereof, in particular geraniol, farnesol, sodium pyruvate, potassium pyrophosphate and the mixtures thereof, such as sodium pyruvate and/or potassium pyrophosphate.

Benzylaminopurine (BAP) can be used, in particular, at a final concentration in the elicitation medium between 0.01 and 5 mg/L, for example between 0.5 and 5 mg/L of elicitation medium. 5-chloro salicylic acid (5-Chloro SA) can be used, in particular, at a final concentration in the elicitation medium between 0.1 and 15 mg/L.

Salicylic acid can be used, in particular, at a final concentration in the elicitation medium between 0.1 and 100 mg/L, for example between 20 and 60 mg/L, for example at approximately 45 mg/L.

Farnesol can be used, in particular, at a final concentration in the elicitation medium between 1 to 100 mg/L, for example between 15 and 30 mg/L, for example at approximately 30 mg/L.

Geraniol can be used, in particular, at a final concentration in the elicitation medium between 1 and 100 mg/L, for example between 20 and 30 mg/L.

Sodium pyruvate can be used, in particular, at a final concentration in the elicitation medium between 100 and 5000 mg/L, for example between 500 and 2000 mg/L. Potassium pyrophosphate can be used, in particular, at a final concentration in the elicitation 30 medium between 1 and 2000 mg/L, for example between 100 and 1000 mg/L of elicitation medium.

6-γ,γ-dimethylallylaminopurine (also called 2iP or isopentenyladenine) can be used, in particular, at a final concentration in the elicitation medium between 0.005 and 10 mg/L, for example between 0.01 and 3 mg/L, for example between 0.1 and 2 mg/L of elicitation medium.

In an embodiment of the invention, the elicitation cocktail comprises methyl jasmonate, chitin, sodium pyruvate, potassium pyrophosphate or a mixture thereof.

In an embodiment of the invention, the elicitation cocktail comprises methyl jasmonate, chitin, sodium pyruvate, potassium pyrophosphate and optionally benzylaminopurine and/or 6-γ,γ-dimethylallylaminopurine.

In an embodiment of the invention, the elicitation cocktail comprises or consists of (the concentrations given between parentheses correspond to the concentration in the elicitation medium, the initial cocktail being able to be more or less concentrated, depending on the envisaged dilution) sodium pyruvate (500 to 2000 mg/L), potassium pyrophosphate (100 to 1000 mg/L), 6-γ,γ-dimethylallylaminopurine (0.1 to 2 mg/L), methyl jasmonate (0.002 to 0.005 g/L) and chitin (1 to 4 g/L).

In an embodiment of the invention, the elicitation cocktail comprises or consists of (the concentrations given between parentheses correspond to the concentration in the elicitation medium, the initial cocktail being able to be more or less concentrated depending on the envisaged dilution) benzylaminopurine (0.5 to 5 mg/L, in particular 0.5 to 3 mg/L), 5-chloro salicylic acid (2 to 6 mg/L, in particular 3 to 5 mg/L, for example approximately 3 or approximately 5 mg/L), acetylsalicylic acid and/or salicylic acid (20 to 60 mg/L, in particular 30 to 50 mg/L, in particular 33 to 45 mg/L), methyl jasmonate (0.002 to 0.05 g/L, in particular 10 to 40 mg/L), chitin (1 to 4 g/L), and farnesol (19 to 40 mg/L) and/or geraniol (20 to 30 20 mg/L).

In an embodiment of the invention, the elicitation cocktail comprises or consists of (the concentrations given between parentheses correspond to the concentration in the elicitation medium, the initial cocktail being able to be more or less concentrated depending on the envisaged dilution) sodium pyruvate (500 to 2000 mg/L), potassium pyrophosphate (100 to 1000 mg/L), 6-γ,γ-dimethylallylaminopurine (0.1 to 1 mg/L), methyl jasmonate (0.002 to 0.05 g/L, in particular 10 to 40 mg/L) and chitin (1 to 4 g/L).

In an embodiment of the invention, the elicitation cocktail comprises or consists of (the concentrations given between parentheses correspond to the concentration in the elicitation medium, the initial cocktail being able to be more or less concentrated depending on the envisaged dilution) sodium pyruvate (approximately 1.5 g/L), potassium pyrophosphate (approximately 0.4 g/L), 6-γ,γ-dimethylallylaminopurine (approximately 0.4 mg/L), methyl jasmonate (approximately 0.03 g/L), and chitin (approximately 2 g/L).

During the elicitation phase, after addition of the elicitation cocktail, the culture is maintained under stirring, in particular between 50 and 200 rpm, in particular at approximately 125 rpm, for a period between 3 and 30 days, in particular between 10 and 25 days, in particular 12 to 15 days, in particular at a temperature between 20 and 35° C., in particular at approximately 27° C. and advantageously with a dissolved oxygen content in the culture medium of 2 to 40%, preferably approximately 16%. A supply of sterile air sufficiently enriched with oxygen can be used, if necessary, in particular in the dead volume of the bioreactor or by diffusion in the medium. The elicitation phase (ii) is preferably performed in the dark. During the elicitation phase, the cell culture is preferably not subcultured.

### Step (iii): Phase of Preparing the Extract Comprising, in Particular Enriched in, Pentacyclic Triterpenes

After the elicitation phase, the method comprises a step of preparing an extract comprising, in particular enriched in, pentacyclic triterpenes.

The extract can, in particular, be obtained by separation of the biomass and the culture supernatant. The separation can, in particular, be made by direct filtration (0-50 μm), by centrifugation or by decanting cells.

In an embodiment, the extract for a use according to the invention can consist of the culture supernatant thus recovered.

The extract can also be obtained after lysis of the biomass. For example, the cells contained in the recovered biomass could be lysed by a physical method (sonication or grinding) or chemical method (acid lysis), then this lysate will undergo extraction by solvent (called extraction solvent). The organic phase, comprising in particular triterpenes from cytosol, is then recovered, in particular by decanting or centrifuging. The solvent will be, in particular, an ester solvent, and more particularly an alkyl acetate, the alkyl being more particularly linear or branched with 1 to 6 carbon atoms, in particular ethyl acetate or isopropyl acetate. The volume of solvent used will be, in particular, 2 volumes per weight of biomass.

Preferably, the extract for a use according to the invention corresponds to the organic phase thus recovered, optionally partially or totally concentrated, i.e. the extraction solvent is partially or totally evaporated.

Alternatively, the extract can be obtained after evaporation of the solvent and optionally its substitution, in particular by a support suitable for the area of use of the extract (cosmetic, pharmaceutical) and in particular by vegetable oils or solvents such as, in particular, pentylene glycol (or pentiol), or Myritol 318®.

The extract thus obtained can optionally be purified in order to obtain a purified extract of one or more pentacyclic triterpenes. The extract according to the invention, and more particularly the purified extract, will advantageously comprise 90% or more, in particular 95% or more, in particular 98% or more by weight of one or more pentacyclic triterpenes according to the invention, relative to the total weight of the dry extract.

In an embodiment, the extract according to the invention, in particular the enriched or purified extract comprises 60% or more, in particular 80% or more, in particular 85% or more, in particular 90% or more, in particular 95% or more, in particular 98% or more, in particular 100% celastrol by weight relative to the total weight of the extract. It can comprise, in particular, between 50 and 98%, in particular between 70 and 90%, in particular between 76 and 84% celastrol by weight relative to the total weight of the dry extract.

In an embodiment, the extract according to the invention, in particular the enriched or purified extract, comprises between 1 and 20%, in particular between 5 and 15%, in particular between 8 and 12% tingenin A by weight relative to the total weight of the dry extract.

In an embodiment, the extract according to the invention, in particular the enriched or purified extract, comprises between 1 and 20%, in particular between 5 and 15%, in particular between 8 and 12% tingenin B by weight relative to the total weight of the dry extract.

In an embodiment, the extract according to the invention, in particular the enriched or purified extract, comprises:
between 50 and 98%, in particular between 70 and 90%, in particular between 76 and 84% celastrol by weight relative to the total weight of the dry extract;
between 1 and 20%, in particular between 5 and 15%, in particular between 8 and 12% tingenin A by weight relative to the total weight of the dry extract;
between 1 and 20%, in particular between 5 and 15%, in particular between 8 and 12% tingenin B by weight relative to the total weight of the dry extract.

The purification of the extract according to the invention can be carried out, in particular, by a phase of separating the one or more pentacyclic triterpenes, in particular celastrol, tingenin A and/or tingenin B, in particular by HPLC (High Performance Liquid Chromatography) fractionation, during which the peaks at 426 nm comprising celastrol, tingenin A and tingenin B emerge respectively at 19.75 minutes, 18.03 minutes and 16.1 minutes.

According to a second aspect, the present invention also relates to a cosmetic or dermatological composition comprising a combination according to the invention, comprising a myrtle extract and an extract of Tripterygium wilfordii, with at least one cosmetically or dermatologically acceptable excipient.

In a particular embodiment, the combination according to the invention is the only active ingredient of the cosmetic or dermatological composition according to the invention.

The invention preferably targets cosmetic or dermatological compositions according to the invention being in a form suitable for a topical application.

The cosmetic or dermatological compositions according to the invention can thus be in the forms which are usually known for a topical application, in other words, in particular, lotions, shampoos, balms, foams, gels, dispersions, emulsions, sprays, serums, masks, creams or sticks with excipients enabling, in particular, for some, penetration in order to improve the properties and accessibility of the active ingredients.

The cosmetic or dermatological composition according to the invention advantageously comprises a myrtle extract which is a non-polar fraction comprising myrtucommulones and ursolic acid. In particular, the myrtucommulones comprise myrtucommulones A, B′, D, B, isosemimyrtucommulmone and semimyrtucommulone.

The cosmetic or dermatological composition according to the invention advantageously comprises an extract of Tripterygium wilfordii which comprises at least one pentacyclic triterpene such as defined above, and in particular chosen from tingenin A, tingenin B, celastrol, pristimerin, tripterygone and the mixtures thereof, and in particular chosen from tingenin A, tingenin B, celastrol and the mixtures thereof, and in particular a mixture of tingenin A, tingenin B and celastrol. The extract of Tripterygium wilfordii will be advantageously an extract of Tripterygium wilfordii comprising, in particular enriched in, pentacyclic triterpene(s), such as tingenin A, tingenin B and/or celastrol, and more particularly an extract of Tripterygium wilfordii comprising, in particular enriched in, pentacyclic triterpene(s) that obtainable by the method described above and which comprises the following steps:
(i) a phase of proliferating Tripterygium wilfordii cells in a proliferation medium,
(ii) an elicitation phase by addition of an elicitation cocktail to the cell culture obtained in step (i), said elicitation cocktail comprising at least one monocarboxylic compound elicitor and at least one biotic elicitor, and
(iii) the preparation of an extract comprising, in particular enriched in, pentacyclic triterpenes from the cell culture obtained in step (ii).

The cosmetic or dermatological composition according to the invention comprises, in particular, between 0.01 and 1% of an extract of Tripterygium wilfordii by weight of dry extract relative to the total weight of the composition, in particular between 0.05 and 0.8%, in particular between 0.1 and 0.5%, in particular between 0.2 and 0.4% by weight of dry extract relative to the total weight of the composition. According to a preferred embodiment, the cosmetic or dermatological composition according to the invention comprises approximately 0.3% of an extract of Tripterygium wilfordii by weight of dry extract relative to the total weight of the composition.

The extract of Tripterygium wilfordii advantageously comprises 90% or more pentacyclic triterpene(s) by weight relative to the total weight of the dry extract. In an advantageous embodiment, the extract of Tripterygium wilfordii comprises between 1 and 20% tingenin A by weight relative to the total weight of the dry extract, between 1 and 20% tingenin B by weight relative to the total weight of the extract and at least 60% celastrol by weight relative to the total weight of the dry extract.

The cosmetic or dermatological composition according to the invention advantageously comprises between 0.01 and 1% of a myrtle extract by weight of dry extract relative to the total weight of the composition, in particular between 0.05 and 0.8%, in particular between 0.08 and 0.4%, in particular between 0.08 and 0.2% by weight of dry extract relative to the total weight of the composition. According to a preferred embodiment, the cosmetic or dermatological composition according to the invention comprises approximately 0.1% of a myrtle extract by weight of dry extract relative to the total weight of the composition.

The myrtle extract advantageously comprises between 3 and 10% myrtucommulones by weight relative to the total weight of the dry extract and/or between 10 and 30% ursolic acid by weight relative to the total weight of the dry extract.

According to a third aspect, the invention relates to a combination according to the invention comprising a myrtle extract and an extract of Tripterygium wilfordii or a cosmetic or dermatological composition comprising such a combination with at least one cosmetically or dermatologically acceptable excipient, for use thereof in the treatment of the inflammation induced by C. acnes.

The invention also relates to a combination according to the invention comprising a myrtle extract and an extract of Tripterygium wilfordii or a cosmetic or dermatological composition comprising such a combination with at least one cosmetically or dermatologically acceptable excipient, for use thereof in the treatment of acne or acne-prone skin.

The following examples illustrate the invention, without limiting the scope thereof.

### DESCRIPTION OF THE FIGURES

FIG. 1 shows the HPLC chromatogram obtained for the “PCC” extract of Tripterygium wilfordii according to example 2 containing the following pentacyclic triterpenes: celastrol, tingenin A and tingenin B.

### EXAMPLES

### Example 1: Preparation of a Myrtle Extract

1 kg of ground myrtle leaves are extracted by 5 volumes of isopropyl acetate under reflux stirring for 1 hour. After filtration and rinsing of the marc, the extraction liquors are bleached by addition of activated carbon. After filtration, the bleached filtrate is concentrated to 2 litres then dried over ethanol until the isopropyl acetate is removed. The aqueous phase obtained is then deodorised by thermal treatment, then dried by lyophilisation.

1 kg de myrtle leaves gives approximately 25 g of dry myrtle extract. This contains 7% myrtucommulones and 25% ursolic acid.

### Example 2: Obtaining a Plant Cell Culture (PCC) Extract of Tripterygium wilfordii Enriched in Pentacyclic Triterpenes

A culture is produced in a Wave reactor (volume 5 L) from Sartorius Stedim Biotech (Germany). The reactor is inoculated with a suspension of Tripterygium wilfordii cells from an Erlenmeyer flask. The proliferation medium has, for example, the composition indicated below:

Macroelements: NH₄NO₃ at 1650 mg/L, KNO₃ at 2500 mg/L, CaCl₂·2H₂O at 440 mg/L, MgSO₄·7H₂O at 370 mg/L, KH₂PO₄ at 130 mg/L;

Microelements: KI at 0.41 mg/L, H₃BO₃ at 6.2 mg/L, MnSO₄·4H₂O at 22.3 mg/L, ZnSO₄·H₂O at 7.5 mg/L, Na₂MoO₄·2H₂O at 0.25 mg/L, CuSO₄·5H₂O at 0.025 mg/L, CoCl_{2·6}H₂O at 0.025 mg/L, FeSO₄·7H₂O at 19.85 mg/L, Na₂EDTA·2H₂O at 26.64 mg/L; Vitamins: myo-inositol at 50 mg/L, nicotinic acid at 0.25 mg/L, pyridoxine hydrochloride at 0.25 mg/L, thiamine hydrochloride at 0.25 mg/L;

Carbon source: sucrose at 30 g/L;

Plant hormones: NAA acid at 0.35 mg/L, 2,4 D acid at 0.575 mg/L, kinetin at 0.083 mg/L.

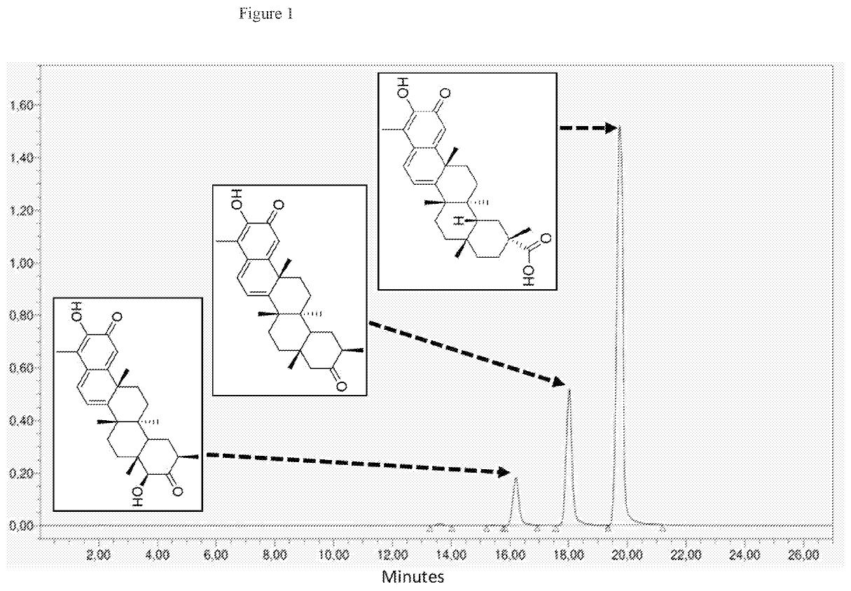

The pH of the medium is adjusted to pH 6±0.5 (by addition of KOH, 1M) before an appropriate sterilisation treatment, for example autoclaving at 121° C. for a minimum duration of 20 minutes or by sterilising filtration at 0.2 μm. Under continuous stirring, after having attained the maximum biomass after approximately 17 days, the elicitation is performed. The elicitation cocktail is then added in the Erlenmeyer flask to the proliferation medium using parent solutions produced in dimethyl sulfoxide. The composition of the elicitor cocktail makes it possible to obtain the following concentrations in the elicitation medium (+cells): sodium pyruvate 1.5 g/L, potassium pyrophosphate 0.44 g/L, 2iP 0.0004 g/L, methyl jasmonate 0.036 g/L and chitin 2 g/L. The culture is stopped after 15 days elicitation. The majority of the biomass is recovered by filtering the cell suspension with a nylon filter (20-50 μm). Approximately 1925 g of biomass are recovered from 5 L of suspension. This biomass is extracted with ethyl acetate (or else with isopropyl acetate) in proportions of 2: 1 (Volume: Weight) relative to the weight of biomass (here 3850 mL of solvent for 1925 g of biomass). The biomass/solvent mixture then undergoes a physical extraction by sonication or by grinding. The organic phase is then recovered after maceration while stirring. The addition of the solvent (following maceration while stifling and recovery of the organic phase) is repeated twice. The solvent is concentrated under vacuum, then the concentrate is solubilised in pentylene glycol, and the mixture placed under vacuum in order to remove the residual organic solvent. A solution, called “PCC” extract, is then obtained, containing the following pentacyclic triterpenes: celastrol, tingenin A and tingenin B, as determined by the HPLC chromatogram shown in FIG. 1.

HPLC chromatography conditions: Alliance liquid chromatography equipment (Waters 2695 version 2.03); Sunfire column C18, 100 Å, 5 μm (4.6 mm×150 mm); water/acetonitrile solvent gradient, flow rate 3 ml/min; detection of triterpenes at λ460 nm.

### Example 3: Biological Properties of the Combination According to the Invention

The aim of this study is to evaluate the anti-inflammatory properties of a myrtle extract and an extract of Tripterygium wilfordii on the inflammation induced by C. acnes organised in a biofilm or by a membrane extract of C. acnes. In order to address this hypothesis, sub-optimal concentrations of myrtle extract, of extract of Tripterygium wilfordii and of their combination are evaluated on the immuno-inflammatory cascade induced in response to the pro-pathogen phylotype of C. acnes (phylotype IA1), upstream of the Th17 path, on the production de pro-inflammatory cytokines by monocytes.

### Method

The experiments were performed on co-cultures of immature dendritic cells derived from monocytes, at a final concentration of 1.10⁵ cells/ml/well and biofilms or membranes of C. acnes of phylotype IA1 . The monocytes are purified from human blood by negative selection (The EasySep™ Human Monocyte Enrichment Kit, StemCell).

On Day 0, the monocytes are incubated in a differentiation medium (Gibco Roswell Park Memorial Institute (RPMI), 10% v/v foetal bovine serum (FBS) that has been decomplemented (heated to 56° C. for 30 minutes), 50 ng/ml IL-4 and 100 ng/ml Recombinant Human Granulocyte Macrophage Colony-Stimulating Factor (GM-CSF)). On the 3^{rd} day of culture, half of the medium is replaced by fresh medium. On day 6, the immature dendritic cells are characterised by flow cytometry and stimulated by a biofilm or membranes of C. acnes.

### Preparation of the Biofilms

### Media Used:

Columbia agar+5% sheep blood (COS)
biofilm broth composed of FeSO₄ 7H₂ O 0.005 g/L+Na₂HPO₄ 12.5 g/L+KH₂PO₄ 5 g/L+vitamin Casamino acids 1g/L+Lactose 0.25 g/L+yeast extract 1 g/L

Preparation of a suspension of C. acnes P52 directly in the biofilm broth at a concentration of order 10⁸ CFU/ml, by adjusting the spectrometer to approximately 54% transmission at 640 nm.

Enumeration of the initial suspension by successive dilutions to 1/10^{th} until the 10⁻⁶ dilution and spreading of 100 μL of the 10⁻⁵ to 10⁻⁶ dilutions on COS agar. Incubation for 72 hours under anaerobic conditions at 36° C.

In order to obtain the biofilm, inoculating all the wells of a 24-well microplate with 2 mL of biofilm broth containing a concentration of C. acnes at 10⁸ CFU/mL, incubation at 36° C. under anaerobic conditions. After 24 hours incubation, renewal of the medium; the wells are emptied then gently rinsed with 2 mL of SDW (sterile distilled water) (once). 2 mL of biofilm broth are introduced into each well and the microplates are return to incubation at 36° C. under anaerobic conditions. After 48 hours incubation, the medium is renewed again under the same conditions.

The biofilms thus prepared are washed with the culture medium (RPMI+10% v/v FBS) before incubation with the dendritic cells.

### Membranes

The membranes of C. acnes are purchased from the Icare laboratory (Saint-Beauzire, France).

The stimulation of the immature dendritic cells derived from monocytes by the membranes of C. acnes is carried out with a dilution to 1/20 of a solution of membranes in the culture medium (RPMI+10% v/v FBS) at a concentration of 0.15 mg/ml.

The stimulation of the dendritic cells derived from monocytes by the biofilms of C. acnes is carried out at a multiplicity of infection (MOI) factor close to 100;

MOI=[C. acnes]/[dendritic cells derived from monocytes].

The myrtle extract used in this study is prepared according to example 1. This extract is solubilised and stored at 20 mg/ml in ethanol (10 mg of dry extract prepared according to example 1, in 1 ml of ethanol). The concentration tested is 10 μg/ml.

The extract of Tripterygium wilfordii (Trip extract below) used in this study is prepared according to example 2. This extract is solubilised and stored at 10 mg/ml in the culture medium (RPMI+10% v/v FBS). The concentration tested is 25 μg/ml.

A positive control for inhibiting the production of inflammatory cytokines is also tested in this study; it involves dexamethasone at a concentration of 300 ng/ml, dissolved in water.

The supernatants are recovered after 24 hours stimulation.

The results are averaged from 3 independent experiments.

Several cytokines are evaluated in this study. IL-6, IL-8, IL-10, and IL-12p40; these cytokines are quantified in multiplex using the Luminex technology (Bioplex 200, Biorad).

### Results

Stimulation by the membranes of C. acnes

The results on inhibition of the production of interleukins 6 (IL-6) by dendritic cells derived from monocytes, stimulated with the membranes of C. acnes are presented in table 1 below.

TABLE 1

| | | | Inhibition | |
|---|---|---|---|---|
| Products | Concentration | | (%) | Statistics |
| Control (C. acnes Mb) | — | | 0 | — |
| Dexamethasone | 300 | ng/ml | 76.8 | P < 0.01 |
| Myrtle extract | 10 | μg/ml | 42.3 | P < 0.05 |
| Trip extract | 25 | μg/ml | 0 | NS |
| Myrtle + Trip extracts | 10 and 25 | μg/ml | 60.3 | P < 0.01 |

Mb: membranes; Trip: Tripterygium wilfordii; NS: not significant.

Without stimulation, the dendritic cells derived from monocytes do not produce IL-6. However, the membranes of C. acnes significantly induce the production of IL-6 by the dendritic cells derived from monocytes. The positive control, dexamethasone at 300 ng/ml, significantly inhibits this production; these expected results allow this test to be validated.

The myrtle extract at 10 μg/ml significantly inhibits the production of IL-6 by the dendritic cells derived from monocytes. The extract of Tripterygium wilfordii at 25 μg/ml does not modify the release of IL-6 relative to the condition of stimulation by the C. acnes membranes.

By contrast, the combination of myrtle and Tripterygium wilfordii extracts at the same concentrations as before, strongly and significantly (p<0.01) inhibits the production of IL-6 with a synergistic effect (Table 1). Indeed, in the presence of these 2 extracts, the inhibition of the production of IL-6 reaches 60%, as opposed to 42% if the effects of the extracts used individually are added together.

The results on inhibition of the production of interleukins 8 (IL-8) by dendritic cells derived from monocytes, stimulated with the membranes of C. acnes are presented in table 2 below.

TABLE 2

| | | | Inhibition | |
|---|---|---|---|---|
| Products | Concentration | | (%) | Statistics |
| Control (C. acnes Mb) | — | | 0 | — |
| Dexamethasone | 300 | ng/ml | 47.6 | P < 0.05 |
| Myrtle extract | 10 | μg/ml | 22.5 | NS |
| Trip extract | 25 | μg/ml | 0 | NS |
| Myrtle + Trip extracts | 10 and 25 | μg/ml | 40.8 | P < 0.05 |

Mb: membranes; Trip: Tripterygium wilfordii; NS: not significant.

Without stimulation, the dendritic cells derived from monocytes do not produce IL-8. However, the membranes of C. acnes significantly induce the production of IL-8 by the dendritic cells derived from monocytes. The positive control, dexamethasone at 300 ng/ml, significantly inhibits this production; these expected results allow this test to be validated.

The myrtle extract at 10 μg/ml alone, is not sufficient to inhibit the production of IL-8 by the dendritic cells derived from monocytes. Similarly, the extract of Tripterygium wilfordii at 25 μg/ml alone does not modify the release of IL-8 relative to the condition of stimulation by the 20 C. acnes membranes. By contrast, the combination of myrtle and Tripterygium wilfordii extracts at the same concentrations significantly (p<0.05) inhibits the production of IL-8 unmasking a synergistic effect (Table 2).

The results on inhibition of the production of interleukins 10 (IL-10) by dendritic cells derived from monocytes, stimulated with the membranes of C. acnes are presented in table 3 below.

TABLE 3

| | | | Inhibition | |
|---|---|---|---|---|
| Products | Concentration | | (%) | Statistics |
| Control (C. acnes Mb) | — | | 0 | — |
| Dexamethasone | 300 | ng/ml | 41.7 | P < 0.01 |
| Myrtle extract | 10 | μg/ml | 42.5 | P < 0.01 |
| Trip extract | 25 | μg/ml | 2.7 | NS |
| Myrtle + Trip extracts | 10 and 25 | μg/ml | 67.2 | P < 0.001 |

Mb: membranes; Trip: Tripterygium wilfordii; NS: not significant.

Without stimulation, the dendritic cells derived from monocytes do not produce IL-10.

However, the membranes of C. acnes significantly induce the production of IL-10 by the dendritic cells derived from monocytes. The positive control, dexamethasone at 300 ng/ml, significantly inhibits this production; these expected results allow this test to be validated.

The myrtle extract at 10 μg/ml significantly inhibits the production of IL-10 by the dendritic cells derived from monocytes. The extract of Tripterygium wilfordii at 25 μg/ml does not modify the release of IL-10 relative to the condition of stimulation by the C. acnes membranes.

By contrast, the combination of myrtle and Tripterygium wilfordii extracts at the same concentrations as before, strongly and very significantly (p<0.001) inhibits the production of IL-10 with a synergistic effect (Table 3). More specifically, in the presence of these 2 extracts, the production of IL-10 is reduced by more than ⅔, as opposed to less than half if the effects of the extracts used individually are added together.

The results on inhibition of the production of interleukins 12p40 (IL-12p40) by dendritic cells derived from monocytes, stimulated with the membranes of C. acnes are presented in table 4 below.

TABLE 4

| | | | Inhibition | |
|---|---|---|---|---|
| Products | Concentration | | (%) | Statistics |
| Control (C. acnes Mb) | — | | 0 | — |
| Dexamethasone | 300 | ng/ml | 79.5 | P < 0.001 |
| Myrtle extract | 10 | μg/ml | 48.3 | P < 0.01 |
| Trip extract | 25 | μg/ml | 9.6 | NS |
| Myrtle + Trip extracts | 10 and 25 | μg/ml | 70.7 | P < 0.001 |

Mb: membranes; Trip: Tripterygium wilfordii; NS: not significant.

Without stimulation, the dendritic cells derived from monocytes do not produce IL-12p40. However, the membranes of C. acnes significantly induce the production of IL-12p40 by the dendritic cells derived from monocytes. The positive control, dexamethasone at 300 ng/ml, significantly inhibits this production; these expected results allow this test to be validated.

The myrtle extract at 10 μg/ml significantly inhibits the production of IL-12p40 by the dendritic cells derived from monocytes, by nearly 50%. The extract of Tripterygium wilfordii at 25 μg/ml alone, tends to reduce the release of IL-12p40, but without attaining significance, relative to the condition of stimulation by the membranes of C. acnes. By contrast, the combination of myrtle and Tripterygium wilfordii extracts at the same concentrations very significantly (p<0.001) inhibits the production of IL-12p40 synergistically (Table 4).

Stimulation by a Biofilm of C. acnes

The results on the production of IL-10 by dendritic cells derived from monocytes, stimulated with biofilm of C. acnes are presented in table 5 below.

TABLE 5

| | | | Inhibition | |
|---|---|---|---|---|
| Products | Concentration | | (%) | Statistics |
| Control (Bf C. acnes) | — | | 0 | — |
| Dexamethasone | 300 | ng/ml | 38.6 | P < 0.05 |
| Myrtle + Trip extracts | 10 and 25 | μg/ml | 59.8 | P < 0.01 |

Bf: biofilm; Trip: Tripterygium wilfordii; NS: not significant.

Without stimulation, the dendritic cells derived from monocytes do not produce IL-10.

However, the biofilms of C. acnes significantly induce the production of IL-10 by the dendritic cells derived from monocytes. The positive control, dexamethasone at 300 ng/ml, significantly inhibits this production; these expected results allow this test to be validated.

The combination of the myrtle (10 μg/ml) and Tripterygium wilfordii (25 mg/nil) extracts inhibits the production of IL-10 even more strongly (p<0.01) than dexamethasone (Table 5).

In conclusion, the inventors have shown that, taken in isolation, the extracts of myrtle and of Tripterygium wilfordii have a relatively modest anti-inflammatory activity in these models by inhibiting the production of various interleukins. By contrast, the inventors have demonstrated a synergy of action for the anti-inflammatory activity, by combining these two extracts.

1. A combination comprising a myrtle extract and an extract of Tripterygium wilfordii.

2. The combination according to claim 1, wherein the myrtle extract is a non-polar fraction comprising myrtucommulones and ursolic acid.

3. The combination according to claim 2, wherein the myrtucommulones comprise myrtucommulones A, B′, D, B, isosemimyrtucommulone and semimyrtucommulone.

4. The combination according to claim 2, wherein the myrtucommulones is present in the myrtle extract in a content between 3% and 10% by weight, relative to the total weight of the myrtle extract in a dry state.

5. The combination according to claim 2, wherein the ursolic acid is present in the myrtle extract in a content between 10% and 30% by weight, relative to the total weight of the myrtle extract in a dry state.

6. The combination according to claim 1, wherein the extract of Tripterygium wilfordii comprises a pentacyclic triterpene.

7. The combination according to claim 6, wherein the pentacyclic triterpene is selected in the group consisting of tingenin A, tingenin B, celastrol, pristimerin, tripterygone and mixtures thereof.

8. The combination according to claim 7, wherein the pentacyclic triterpene is selected in the group consisting of tingenin A, tingenin B, celastrol and mixtures thereof.

9. The combination according to claim 6, wherein the extract of Tripterygium wilfordii is obtainable by:
(i) placing Tripterygium wilfordii cells in a proliferation medium for inducing a proliferation phase in order to obtain a first culture cell,
(ii) adding an elicitation cocktail to the first cell culture obtained in step (i) for inducing an elicitation phase in order to obtain a second culture cell, wherein the elicitation cocktail comprises a monocarboxylic compound elicitor and a biotic elicitor, and
(iii) preparing the extract of Tripterygium wilfordii comprising the pentacyclic triterpene from the second cell culture obtained in step (ii).

10. The combination according to claim 6, wherein the extract of Tripterygium wilfordii comprises 90% by weight of the pentacyclic triterpene relative to the total weight of the extract of Tripterygium wilfordii in a dry state.

11. The combination according to claim 10, wherein the extract of Tripterygium wilfordii comprises between 1% and 20% tingenin A by weight, relative to the total weight of the extract of Tripterygium wilfordii in a dry state, between 1% and 20% tingenin B by weight relative to the weight of the extract of Tripterygium wilfordii in a dry state and at least 60% celastrol by weight relative to the total weight of the extract of Tripterygium wilfordii in a dry state.

12. (canceled)

13. (canceled)

14. A cosmetic or dermatological composition comprising a combination according to claim 1 with at least one cosmetically or dermatologically acceptable excipient.

15. The composition according to claim 14, comprising 0.01% to 1% of the extract of Tripterygium wilfordii in a dry state by weight relative to the total weight of the composition.

16. The composition according to claim 14, comprising 0.01% to 1%, by weight of the myrtle extract in a dry state relative to the total weight of the composition.

17. The composition according to claim 14, being in a form suitable for a topical application.

18. (canceled)

19. (canceled)

20. A method for treating an inflammation induced by C. acnes comprising the administration to a person in need thereof of an effective amount of the combination according to claim 1.

21. A method for treating an acne-prone skin comprising the administration to a person in need thereof of an effective amount of the combination according to claim 1.

22. A method for treating an inflammation induced by C. acnes comprising the administration to a person in need thereof of an effective amount of the composition according to claim 14.

23. A method for treating an acne-prone skin comprising the administration to a person in need thereof of an effective amount of the composition according to claim 14.
